(19) United States
(12) Patent Application Publication
(10) Pub. No.: US 2019/0083003 A1
(43) Pub. Date: Mar. 21, 2019

(54) HEALTHCARE CAREGIVER BEHAVIOR COACHING SYSTEM AND METHOD

(71) Applicant: Johnson & Johnson Consumer Inc., Skillman, NJ (US)

(72) Inventors: Christina Irene Lee, Plainsboro, NJ (US); Janeta Nikolovski, Princeton, NJ (US); Russel M. Walters, Philadelphia, PA (US); Carson James Darling, Cambridge, MA (US); Thomas Samuel Lipoma, Boston, MA (US); Dulcie Merrill Madden, Boson, MA (US)

(21) Appl. No.: 16/135,322

(22) Filed: Sep. 19, 2018

Related U.S. Application Data
(60) Provisional application No. 62/560,938, filed on Sep. 20, 2017.

(51) Int. Cl. A61B 5/113; A61B 5/024; A61B 5/00

(52) U.S. Cl. CPC A61B 5/113; A61B 5/024; A61B 5/01; A61B 5/4809; A61B 5/0004; A61B 5/4812

(57) ABSTRACT

A health care system for a caregiver to monitor and manage sleep metric, patterns and quality for an infant, including: a) a base station in communication with a network, b) one or more sensors in communication with the base station, c) a caregiver communication device in communication with the network; and d) a remote server and associated data store in communication with the network. The remote server is operative to: 1) access information from the information store indicating caregiver typing traits, 2) receive information from the sensors indicating a sleep quality for the infant, 3) receive information from the caregiver communication device indicating a caregiver perception of sleep quality for the infant, 4) recommend at least one caregiver action as a function of the caregiver typing traits, the sleep quality measures and the caregiver perception of the sleep quality; and 5) transmit the recommended action to the caregiver communication device.

### FIELD OF THE INVENTION

This disclosure pertains to a system and method for coaching actions taken by caregivers, and more particularly, to a system and method for coaching caregivers managing the daily routines of infants, for example, as these routines may influence sleep quality characteristics including for example circadian rhythms and diurnal patterns.

### BACKGROUND

For new parents, managing baby sleep is an important and high priority and need. Common areas for need and questions of parents include: getting their babies to fall asleep at bedtime, establishing a sleep routine or schedule, and helping their babies stay asleep through the night.

Managing baby's sleep is inherently a hard problem. When babies are first born, they have not yet developed circadian rhythms, and do not distinguish night from day. As such, in the early years of life, babies sleep at all times (both night and day), and usually in short bouts. Typically, new parents are strongly motivated to help their babies sleep through the night, and often seek assistance in obtaining such outcomes.

U.S. Published Application No. 20150094830 A1 to Rest Devices, Inc. (“Network-Based Care System”), which is hereby incorporated by reference in its entirety herein, discloses a computerized health/sleep monitor that monitors biometric data of an infant to determine infant conditions relating to sleep quality (for example, such as the infant being awake or asleep, the infant being irritated, fussy or crying, or the infant being hungry). The monitor sends associated information via a network to an event server that evaluates whether or not to alert a caregiver via a caregiver's personal communication device (for example, via the caregiver's mobile phone, personal computer or tablet device)

WO2017196695 to Udisense, Inc. discloses a video monitoring system configured to hold a camera head in a fixed location and orientation above a crib.

U.S. Published Application No. 20160293042 to Smilables, Inc. discloses mechanisms and processes for monitoring an infant's emotional state. In one example, a system includes an infant monitoring hub that has an infant monitoring device interface and a hub processor. The infant monitoring device interface receives measurement data transmitted wirelessly from an infant monitoring device associated with a first infant. The hub processor compares the measurement data to a development model to determine if an emotional state associated with the measurement data reaches an undesirable level and generates a notification for a caregiver associated with the first infant if the emotional state reaches an undesirable level.

U.S. Published Application No. 20170043118 to Happiest Baby, Inc. discloses a sleep-aid device that includes a main moving platform that moves in a variable manner with accompanying variable sound generation, the sound and motion under the control of a control system and adapted to calm a fussy baby, induce sleep, and maintain sleep under normal conditions.

U.S. Published Application No. 20170055898 to Awardables, Inc. discloses systems and apparatus, including computer programs encoded on a computer storage medium, for determining sleep stages and sleep events using sensor data.

U.S. Pat. No. 8,562,511 to Koninklijke Philips N.V. discloses a system for inducing a subject to fall to sleep that includes a control unit connected to a breathing rate measuring unit and a light pattern generator, for controlling the light pattern generator such that the generated light pattern has a pattern frequency substantially between the measured breathing frequency and a pre-selected desired frequency.

U.S. Pat. No. 8,532,737 to Cervantes discloses an apparatus for automatically monitoring sleep, including a video recorder for recording live images of a subject sleeping, including a transmitter for transmitting the recorded images in real-time to a mobile device, and a computing device communicating with the transmitter, including a receiver for receiving the transmitted images in real-time, a processor for analyzing in real-time the received images and for automatically inferring in real-time information about the state of the subject, and a monitor for displaying in real-time the information inferred by said processor about the state of the subject.

U.S. Pat. No. 9,530,080 to Joan and Irwin Jacobs Technion-Cornell Institute discloses systems and methods for monitoring babies with cameras using a centralized computation and storage center that allows using visual output signals for computer vision and machine learning analysis and high-level reasoning of baby movements.

U.S. Pat. No. 9,572,376 to Nested Bean Inc. discloses a wearable or swaddling accessory blanket provides gentle pressure on the side and/or on the thoracic area of an infant to mimic the human hold.

Upon receiving such alerts, a caregiver may experience anxiety in attempting to determine whether caregiver action is needed, and if so, what actions would be most appropriate and effective for meeting caregiving goals. Accordingly, it would be beneficial to provide caregivers with specific advice that is directed to meeting their caregiver goals and well-matched to their individual preferences and tendencies in order to minimize caregiver anxiety.

### SUMMARY

By way of example, aspects of the present disclosure are directed to a health care system and method for coaching a caregiver that monitors and manages sleep quality for an infant. The caregiver may be a parent, grandparent, guardian or other individual responsible for the health and well-being of the infant.

According to aspects of the present disclosure, the health care system described herein preferably includes: a) a base station in communication with a network, b) one or more sensors in communication with the base station that are configured to monitor sleep-relevant characteristics of the infant and environmental conditions in proximity to the infant, c) a caregiver communication device in communication with the network; and d) a remote server and associated data store in communication with the network. The remote server is operative to: 1) access information from the information store indicative of caregiver typing traits for the caregiver, 2) receive information from the sensors via the base station indicative of one or more measures of sleep quality for the infant, 3) receive information from the caregiver communication device indicative of a caregiver perception of sleep quality for the infant, 4) recommend at least one action to be taken by the caregiver as a function of the caregiver typing traits, the sleep quality measures and the caregiver perception of the sleep quality for the infant; and 5) transmit the recommended action to the caregiver communication device for execution by the caregiver.

According to another aspect of the present disclosure, the remote server may thereafter be preferably operative to: a) confirm that the recommended caregiver action was applied, b) receive updated information from the sensors indicative of one or more measures of a current sleep quality for the infant, c) receive updated information from the caregiver communication device indicative of a current caregiver perception of sleep quality for the infant, d) receive an updated caregiver perception of the sleep quality for the infant; and e) evaluate the effectiveness of the recommended action in improving the caregiver's perception of sleep quality.

This SUMMARY is provided to briefly identify some aspects of the present disclosure that are further described below in the DESCRIPTION. This SUMMARY is not intended to identify key or essential features of the present disclosure nor is it intended to limit the scope of any claims.

### BRIEF DESCRIPTION OF THE DRAWINGS

A more complete understanding of the present disclosure may be realized by reference to the accompanying drawing in which:

FIG. 1 depicts a health care system according to aspects of the present disclosure;

FIGS. 2A and 2B illustrate information flows for setting caregiver goals and providing caregiver advice according to aspects of the present disclosure;

FIG. 3 provides examples of data gathered in support of input state variables according to aspects of the present disclosure;

FIGS. 4A and 4B provide schematic diagrams illustrating information flows in a health care system according to aspects of the present disclosure;

FIG. 5 provides a schematic diagram illustrating a flow of information in a health care system according to additional aspects of the present disclosure;

FIGS. 6A and 6B provide an illustration of a daily routine builder for managing infant sleep quality interventions, and a related schedule of behavioral quanta;

FIG. 7 depicts element of large data set modeling of infant and caregiver behaviors in accordance with aspects of the present disclosure;

FIG. 8 depicts a data assimilation hierarchy for managing the large data set modeling depicted in FIG. 7; and

FIG. 9 depicts an analysis engine for analyzing the data in the data hierarchy of FIG. 8.

FIG. 10 shows the relative change in baby sleep score after use of the infant digital sleep coaching system of the invention.

FIG. 11 shows the significance of the results of baby sleep score after use of the infant digital sleep coaching system of the invention.

### DETAILED DESCRIPTION

The following merely illustrates the principles of the disclosure. It will thus be appreciated that those skilled in the art will be able to devise various arrangements which, although not explicitly described or shown herein, embody the principles of the disclosure and are included within its spirit and scope.

Furthermore, all examples and conditional language recited herein are principally intended expressly to be only for pedagogical purposes to aid the reader in understanding the principles of the disclosure and the concepts contributed by the inventor(s) to furthering the art, and are to be construed as being without limitation to such specifically recited examples and conditions.

Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements later developed that perform the same function, regardless of structure.

Unless otherwise explicitly specified herein, the drawings are not drawn to scale.

In accordance with aspects of the present disclosure, a health care system and method are disclosed for assisting a caregiver who is tasked, for example, to monitor sleep quality for an infant. The caregiver may in this case be a parent, grandparent, day care worker or any other person tasked with monitoring and influencing the sleep quality for the infant. The health care system and method may, for example, be directed to multiple caregivers having sequential or simultaneous responsibility over a defined time period for providing infant care.

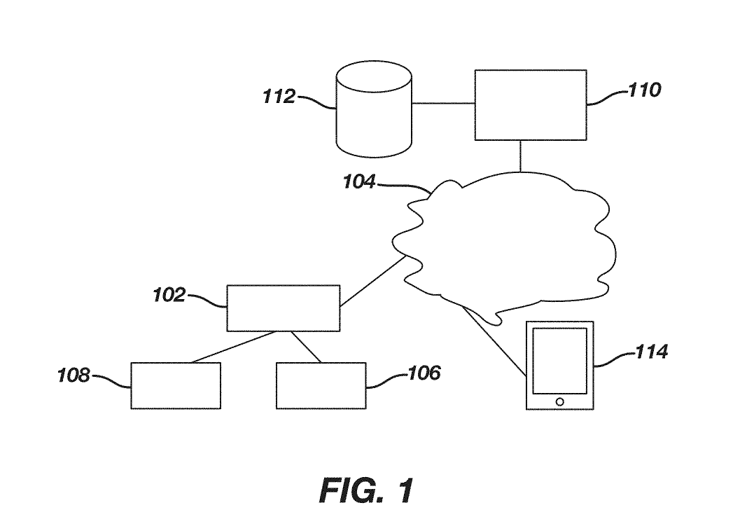

FIG. 1 presents a high level schematic diagram illustrating a health care system according to aspects of the present disclosure. The system of FIG. 1 includes a base station 102 in communication with a network 104, and also in communication with biometric sensor(s) 106 for monitoring certain biologic condition(s) of the infant and environmental sensor(s) 108 for monitoring certain environmental conditions in proximity to the infant (not shown). Biologic conditions may, for example, include heart and breathing rate, movement and other sleep-related indicators useful for determining whether the infant is asleep, awake, irritated, fussy, crying and so on. The environmental conditions, for example, may include temperature, sound types and levels, light coloration, patterns and intensity, odors and other indicators useful for influencing a state of the infant. A suitable base station and sensor configuration for this purpose may be obtained, for example, from Rest Devices, Inc. of Boston, Mass. See, e.g., U.S. Published Applications Nos. 20130197387; 20140213937 (now abandoned); 20150060720 (now abandoned); 20150105608 (now abandoned); and 20150094830.

A remote server 110 is also in communication with the network 104, and may be operative for example to access information stored in an information store 112 indicating one or more caregiver typing traits for at least one caregiver. The remote server 110 receives information from the sensors 106, 108 via the base station 102 to be interpreted as indicating one or more measures of sleep quality for the infant,

The caregiver is also able by means of a caregiver communication device 114 to communicate with the remote server 110 via the network 104. For example, the caregiver communication device 114 may be by a smartphone, tablet computer, personal computer or other device that can be identified to the caregiver and be configured to communicate with the network 104. The caregiver may, for example, communicate with the remote server 110 via the network 104 or another alternate network to provide a caregiver perception of sleep quality for the infant.

Based on the stored caregiver typing traits, the biologic and environmental conditions, the caregiver perception and certain goals of the caregiver with respect to the infant's sleep quality, the remote server 110 is operative to recommend at least one action to the caregiver to be taken in support of managing or improving infant sleep quality.

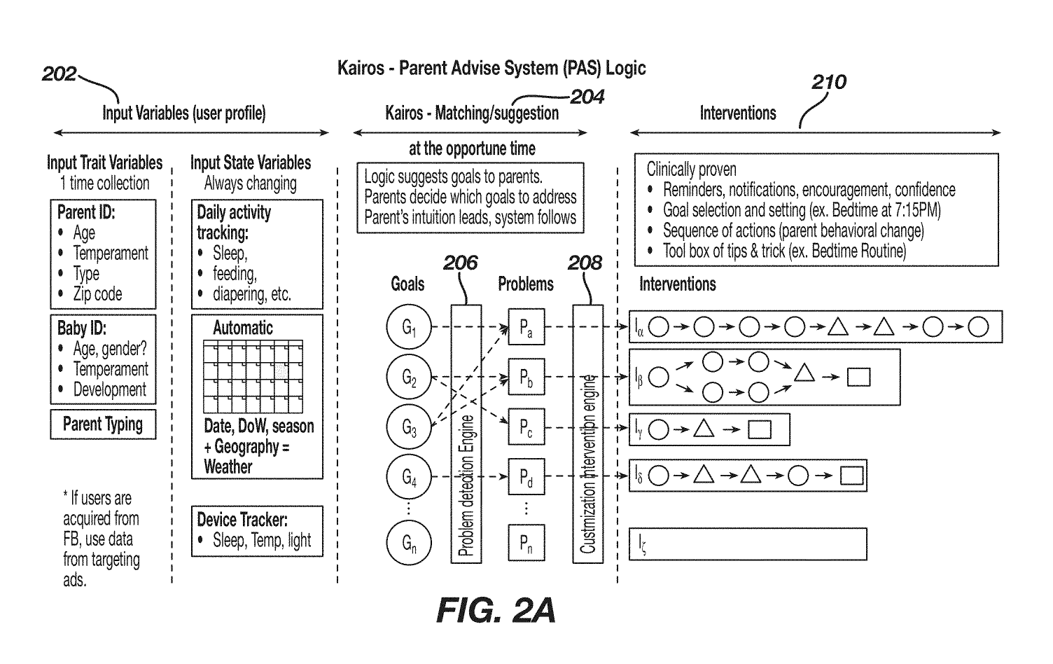

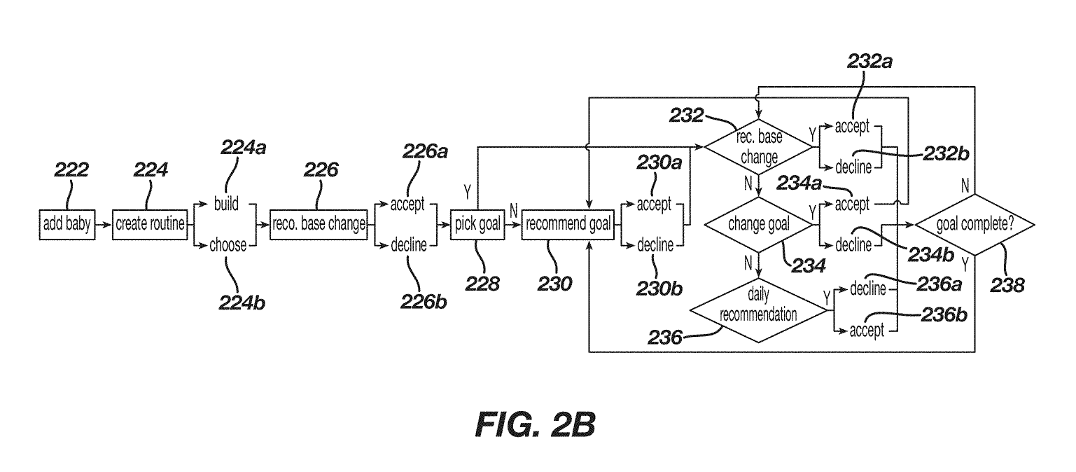

FIG. 2A illustrates an information flow for the remote server 110 according to aspects of the present invention. Server 110 begins by establishing certain input variables at step 202 that pertain to the infant and the caregiver. For example, the server 110 may collect information to identify the caregiver (for example, a parent) according to age, general temperament, and location (for example, zip code). The infant may be similarly characterized by age, gender, temperament and developmental stage. This information may be referred to generally as identified input trait variables, which are static and require collection once or only infrequently.

In addition to the input trait variables, certain information indicative of daily activities of the infant (for example, sleep, feeding and diapering) may be gathered together with information about the season, geography and weather, and local environmental conditions (for example, temperature and light profiles) via the base station 102 and sensors 106, 108, This information may be referred to collectively as identified input state variables, which are dynamic and require ongoing, periodic collection.

An important category of input trait variables is directed to parent or caregiver typing variables. These are used to characterize different groups of caregivers according to the kinds of infant care interventions they may be comfortable and capable of providing, thereby increasing the likelihood that interventions coached by the inventive system will be carried out by the caregivers. In one embodiment in accordance with the present disclosure, caregiver typing is accomplished by causing the remote server 110 to transmit and administer a caregiver questionnaire to the caregiver via the caregiver communication device 114. Information indicative of the answers that the caregiver provides to the questionnaire are stored by the remote server 110 in the information store 112. As illustrated below, the questions administered to determine caregiver typing variables may preferably be provided with discrete answers (“options”) to facilitate easy compilation by the remote server 110:

| Name | Question | options |
|---|---|---|
| FA1 | Where does your child sleep | Own Room, |
| | most of the time? | Parent's Room, |
| | | Sibling's or others room, |
| | | Other room of the house |
| FA2 | What does your child sleep in | Crib, Own bed (any |
| | most of the time? | size), Parent's bed, |
| | | Bassinet/Seat/Swing |
| FA3 | Does your child usually fall asleep | Yes, No |
| | while being fed (breast or bottle)? | |
| FA4 | How does your child fall sleep | While being rocked or |
| | most of the time? | held, In a swing of |
| | | stroller, While watching |
| | | television, |
| | | On his/her own |
| FA5 | Where does your child fall asleep? | In own bed, In parents |
| | | bed, Crib |
| SR1 | How often does your child have the | Never, 1-2 nights |
| | same bedtime routine? | per week, 3-4 nights |
| | | per week, 5-6 nights |
| | | per week, Every night |
| SR2 | What time do you start your child's | clock time: |
| | bedtime routine? | XX:XX AM/PM |
| SD1 | What is your child's bedtime | clock time: |
| | (lights out)? | XX:XX AM/PM |
| NW3 | Do you usually feed (breast of bottle) | Yes, No |
| | your child when s/he | |
| | wakes up at night? | |
| NW4 | Do you usually give your child a | Yes, No |
| | pacifier when s/he wakes up at night? | |
| NW5 | Do you usually change your child's | Yes, No |
| | diaper when s/he wakes up at night? | |
| NW6 | Do you usually play or watch | Yes, No |
| | TV/Video games? | |
| NW7 | Do you usually pick up, pat, or bring | Yes, No |
| | your child to your bed when s/he | |
| | wakes up at night? | |
| SRc | (SD1-SR2) | time: XX Hr: |
| | | XX Min |

Examples of additional caregiver typing questions are provided in Appendix 1. The aforementioned questions, those in Appendix 1, and those set forth below may be referred to generally as “metrics”, or “sleep metrics”, and may include or demonstrate quality of sleep. “Patterns” may reflect how the questions, or a subset of these questions occur and/or reoccur throughout the course of a day, and from day to day.

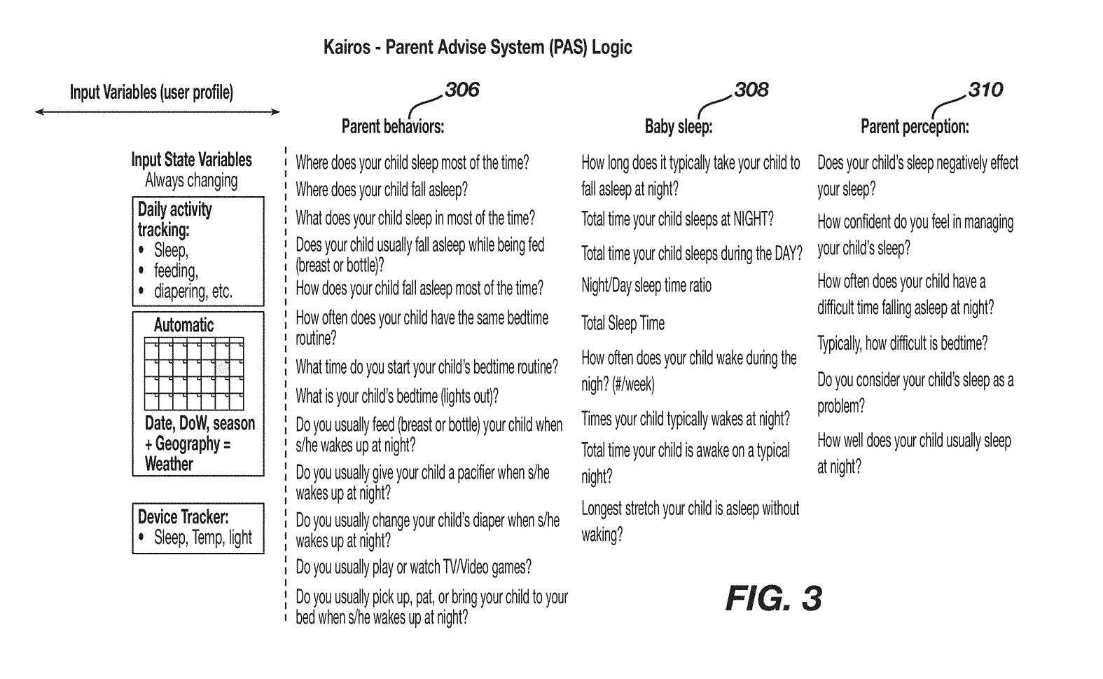

Examples of information gathered for determining the second category of variables (input state variables) are illustrated in FIG. 3. Information gathered may include tracking of the infant's daily activities (sleeping, feeding, diapering and so on); information about the date, season, day of the week and weather; and information about environmental conditions proximate to the infant (for example, temperature, sound and light). The significance of these variables can be evaluated, for example by querying the caregiver with regard to associated caregiver behaviors 306, baby sleep parameters 308, and caregiver perception factors 310. As illustrated below, the questions administered to determine baby sleep parameters 308 may preferably be provided with discrete answers to facilitate easier compilation by the remote server 110:

| Name | Question | options |
|---|---|---|
| SOL | How long does it typically take your | time: XX Hr: XX Min |
| | child to fall asleep at night? | |
| NTS | Total time your child sleeps at NIGHT? | time: XX Hr: XX Min |
| DTS | Total time your child sleep during the | time: XX Hr: XX Min |
| | DAY? | |
| NW1 | How often does your child wake during | 3 nights a week |
| | the night? (#/week) | or more, Less than |
| | | 3 nights a week, Never |
| NW2 | Times your child typically wakes at | 0, 1, 2, 3, 4, 5 or more |
| | night? | |
| NW8 | Total time your child is awake on a | time: XX Hr: XX Min |
| | typical night? | |
| NW9 | Longest stretch your child is asleep | time: XX Hr: XX Min |
| | without waking? | |
| TSTc | (NTS + DTS) | time: XX Hr: XX Min |
| NDRc | (NTS/DTS) | time: XX Hr: XX Min |

Returning to FIG. 2A, the server 210 employs a goal/problem-driven approach (i.e., the right or opportune moment, “Kairos”) 204 to determine suitable interventions to be taken by a caregiver to improve infant sleep quality. Caregivers begin by setting goals for infant sleep quality. A problem detection engine 206 correlates the goals with known problems based on an analysis of the input variables 202. Caregivers prioritize the goals based, for example, on their intuition, beliefs and individual preferences. For high priority goals and high impact problems, a customization intervention engine 208 selects certain interventions (for example, reminders, notifications, and encouraging messages) for action by the caregiver.

FIG. 2B further illustrates an exemplary process by which goals evolve. At step 222, a new infant is added to a goal setting regime, which may be implemented for example as a software-guided planning system. An infant routine is created at step 224, either built by scratch at step 224a or based on existing templates at step 224b. Base on the selected routine, a base change to an element of the routine is recommended at step 226. The caregiver may accept or decline this recommendation at steps 226a, 226b, respectively. At step 228, the caregiver may select a particular goal, or rely on the system to suggest a goal at step 230. The caregiver accepts or rejects the recommended goal at steps 230a, 230b, respectively.

Once a goal is selected, a further base change is recommended by the system at step 232, which can be accepted or declined at steps 232a, 232b, respectively. If not accepted, the system suggests a goal change at step 234, which can be accepted or declined at steps 234a, 234b respectively. If the change is not accepted, the system may recommend a daily objective as an alternative at step 236, which can be accepted or declined at steps 236a, 236b, respectively. If the goal has been completed at step 238, system returns to step 230 to suggest a new goal. Otherwise, the system returns to step 232 to recommend a further base change.

Recommended changes may stimulate a variety of actions to adjust the infant's environment and routine, for example, such as:

Set/encourage consistent bedtime:
Light source changes lighting—intensity, wavelength, direction change over the course of the day.
Speaker turns on/changes sounds—Specific songs or ambient sounds are triggered or play at bedtime.

Set/encourage consistent bedtime routine:
Physical chart in baby's room with bedtime routine check list—for reminder of instructions for additional care givers
Baby bath product as part of bedtime bath
Baby lotion product as part of bedtime massage
Books are part of bedtime routine—books that promote positive sleep messages. Many children's books themes involve resistance to sleep—not a positive bedtime message
Release of fragrance at specific time (or human triggered).

Help child self sooth back to sleep at night-triggered events that the system does automatically after noticing that the baby has waken in the middle of the night
Auto pacifier dropped into crib.
Speaking that play care-givers voice, songs or ambient sounds.
Movement of mobiles
Release of fragrance

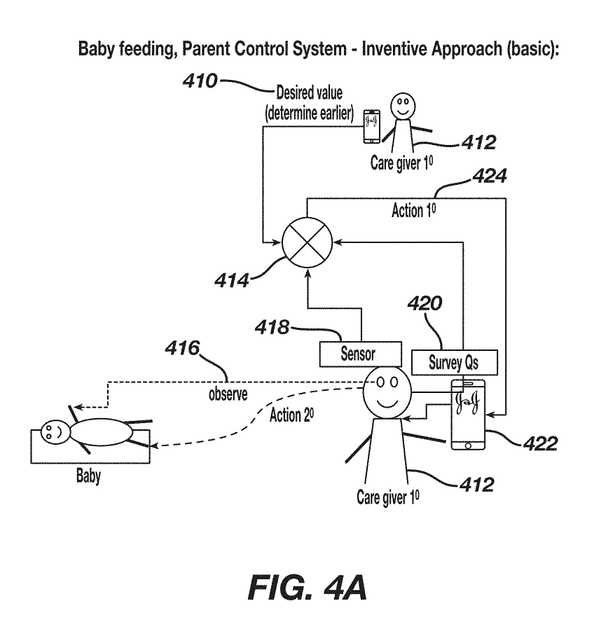

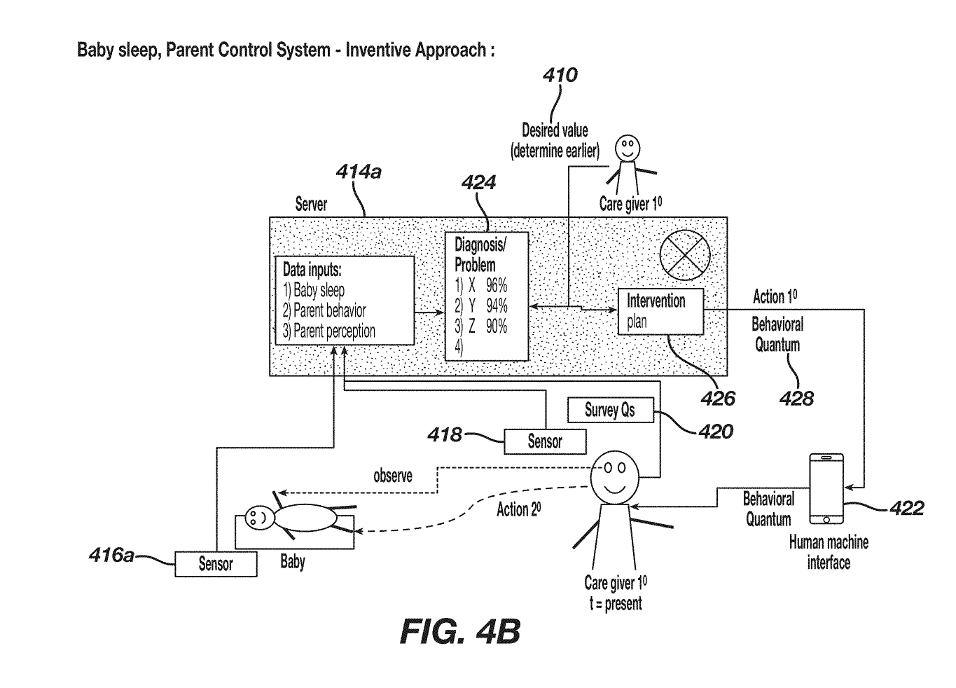

FIG. 4A provides a schematic diagram illustrating a flow of information in a health care system described in accordance with aspects of the present disclosure; A control characteristic or condition 410 is established by a primary caregiver 412, for example, by the goal/problem-driven approach 204 of FIG. 2. A comparator 414 (implemented, for example, as the remote sever 110 of FIG. 1) compares the control characteristic 410 with an observation 416 of the primary caregiver 412, who acts effectively as a sensor 418 and provides the observation to the comparator 414 by answering a series of survey questions 420 presented at a caregiver communication device 422. The comparator (for example, realized by the remote server 110 of FIG. 1), applies the goal/problem-driven approach 204 of FIG. 2 to determine a caregiver intervention to be instructed through the caregiver communication device 422 as action 424. This cycle is repeated while the observation 416 by the caregiver 412 indicates a deviation from the control characteristic 410.

Comparator 414 is further illustrated as implemented by server 414a in FIG. 4B. Server 414a gathers biometric and other sensory data via sensor(s) 416(a) placed in the vicinity of the infant, which may for example be used to determine infant sleep patterns. Server 414a also gathers data from sensor(s) 418 that indicate(s) parent or caregiver behaviors (for example, such as parent responses to queries administered by the caregiver communication device 422 to determine whether recommended interventions were administered, and parent location and movement data provided by GPS sensors incorporated in the caregiver communication device 422). Sensor(s) 418 may also provide data indicative of parent or caregiver perceptions of care (for example, via surveys administered via the caregiver communication device 422).

By means further described with reference to FIGS. 4A and 4B herein, the server 414a applies the data inputs gathered by the server 414a to produce a probabilistic diagnosis 424 of potential problems which may for example be preventing infant sleep characteristics and parent perception from reaching values consistent with the identified goals. Through further analysis of data as described by way of example with reference to FIGS. 4A and 4B, server 414a selects an intervention plan 426 including one or more behavioral quanta 428 expressed as actions to be taken by the parent or caregiver for the purpose of carrying out an intervention. These behavioral quanta or actions may be displayed, for example, to the caregiver via the caregiver communication device 422. In this “closed loop” system, actions taken by the caregiver influence infant sleep characteristics in a direction towards or away from desired values and goals 410, thereby providing a basis for adjusting the associated intervention plan 426 and behavioral quanta 428.

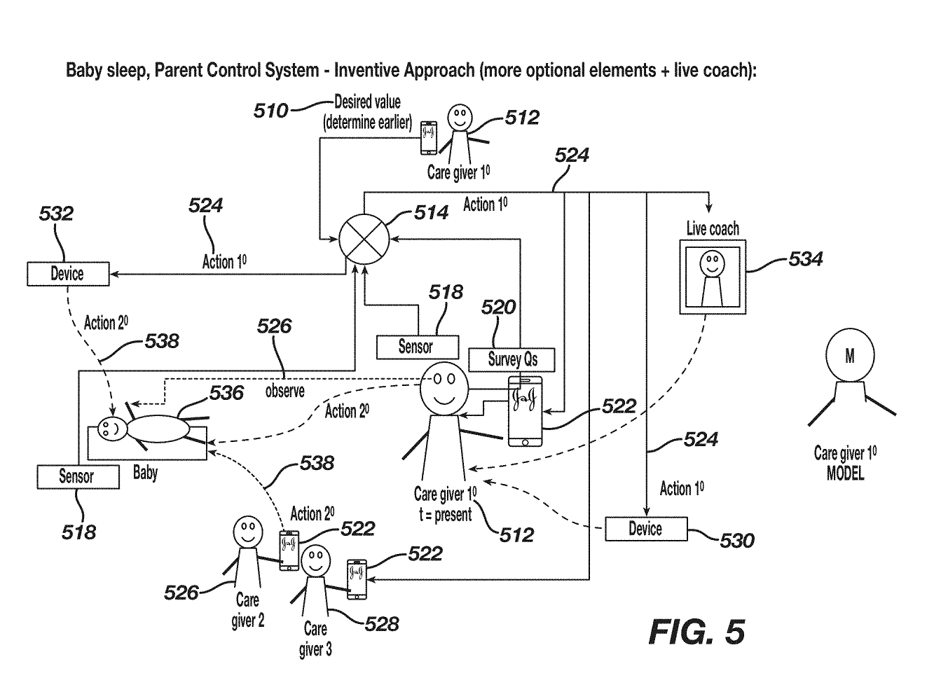

In accordance with additional aspects of the present disclosure, FIG. 5 provides a schematic diagram illustrating an alternate flow of information in the described health care system. A control characteristic or condition 510 is established by a primary caregiver 512, for example, by the goal/problem-driven approach 204 of FIG. 2. A comparator 514 (implemented, for example, as the remote sever 110 of FIG. 1) compares the control characteristic 510 with an observation 516 of the primary caregiver 512, who acts effectively as a sensor 518 and provides the observation to the comparator 514 by answering a series of survey questions 520 presented at a caregiver communication device 522. Additional sensors 518 (for example, including one or more of biometric sensors 106 and environmental sensors 108 of FIG. 1) are provided in proximity to an infant 532 for assessing characteristics indicative of or capable of influencing infant sleep quality.

The comparator 514 (again realized, for example, by the remote server 110 of FIG. 1), applies the goal/problem-driven approach 204 of FIG. 2 to determine a caregiver intervention to be instructed through the caregiver communication device 522 as action 524. In addition, action 524 may be instructed to devices 530, 532 for implementation. For example, if the action 524 is intended to be performed by primary caregiver 512 immediately after the primary caregiver wakes from a period of sleep, device 530 may be provided in proximity to primary caregiver 512 to open curtains to sunlight, to play certain audio sounds that are wakeful, or the like. Device 530 may be provided in proximity to the infant 532 for performing an action 538 that may support a caregiver intervention (for example, providing the infant 532 with a pacifier or initiating the movement of a mobile in combination with a soothing sound). For example, if the action 524 instructs the primary caregiver 512 to rock the infant 530 in order to urge the infant 532 to cease crying, action 538 may in addition be applied in advance by device 532 in support of caregiver action 524. This cycle continues to be repeated while the observation 516 by the caregiver 512 indicates a deviation from the control characteristic 510.

In addition to primary caregiver 512, secondary caregivers 526, 528 may assist primary caregiver 512 concurrently with primary caregiver 512 or at alternate times when primary caregiver 512 is unavailable, and be provided with caregiver communication devices 522 to receive instructions concerning caregiver interventions. Secondary caregivers 526, 528 will most likely be taking action directed to the control characteristics 510 established by primary caregiver 512. For example, in order to progress to a control characteristic 510 that is intended to encourage infant sleep after 8:00 PM in the evening, secondary caregivers 526, 528 may undertake a supporting action 538 directed to bathe the infant 532 at 7:00 PM.

Secondary caregivers 526, 528 may have parent typing characteristics that differ from the primary caregiver 512. For example, this might be expected in the case where primary caregiver 512 is a parent of the infant 532, and secondary caregivers 526, 528 are grandparents of the infant 532. With reference to FIGS. 2 and 3, parent typing may therefore be preferably performed by administering separate surveys to each of the primary caregiver 512 and secondary caregivers 526, 528 to account for differences in caregiving tendencies and styles among the various caregivers. In this case, customization intervention engine 208 of FIG. 2 may select interventions 210 that are accordingly tailored to the caregiving tendencies and styles of each on-duty caregiver.

As illustrated in FIG. 5, in addition to actions 524 that may be instructed at caregiver communication device 522 as text-based instructions, notifications and reminders, action 524 may be instructed at caregiver communication device 522 by a “live” human coach (for example, by means of direct a FACETIME, SKYPE or other audio/video link), or alternatively by means of an interactive avatar that is animated by remote server 110 of FIG. 1. Some caregivers may find they are more at ease with this approach to receiving intervention instruction and additional guidance than with text-based instructions. The avatar may be implemented as an available array of many adviser/expert avatars (in effect, a “Many Face God” engine) having distinct styles, selectable to match with the caregiver's typing characteristics.

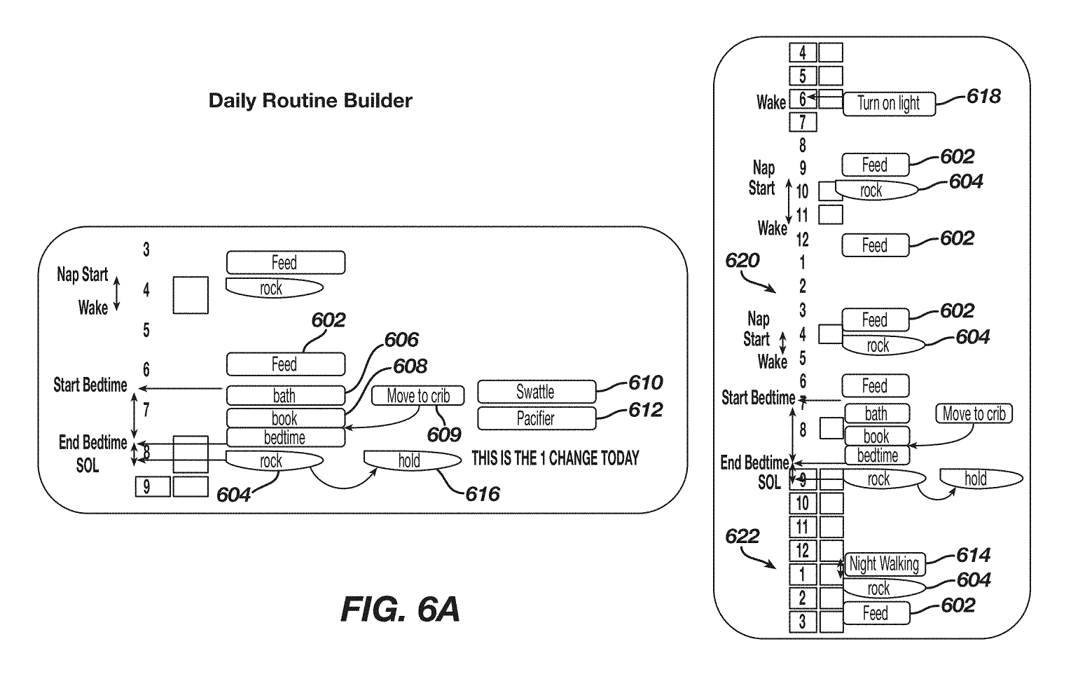

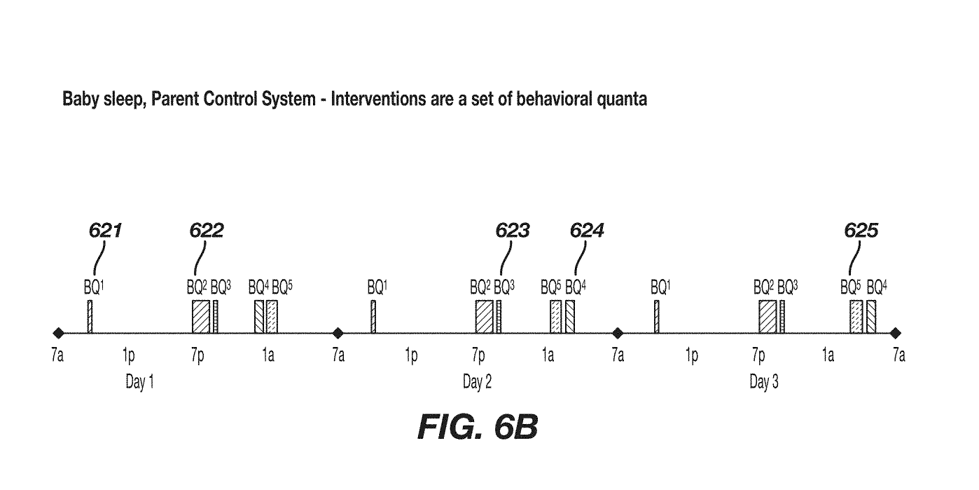

FIG. 6A provides an illustration of a daily routine builder for managing infant sleep quality interventions according to aspects of the present disclosure. Experience suggests that a managed routine is essential to stabilizing and promoting good infant sleep quality. In accordance with aspects of the present disclosure, the primary caregiver is able to assemble and record a daily routine with the assistance of the remote server 110 of FIG. 1 via the caregiver communication device 114. For example, as illustrated in FIG. 6A, tasks that may be repeatedly scheduled over the course of a day and over successive days may include feedings 602, rockings 604, bathings 606, and readings 608. Daylight timeline 621 and night timeline 622 (FIG. 6B) provide a baseline against which the tasks may be arrayed and scheduled.

The daily routine builder may preferably include transitional tasks to assist the infant in moving from one state to another. For example, FIG. 6A illustrates task 618, which instructs the caregiver to turn on a light in proximity to a sleeping infant at 6:00 AM to assist in moving the infant from a sleeping to wakeful state. Similarly, Task 614 instructs the caregiver to move the infant from a sleeping to a wakeful state at 1:00 AM in order to administer rocking 604 and feeding 602.

As further illustrated in FIG. 6A, the remote server 110 of FIG. 1 may instruct the primary caregiver to alter a pre-existing daily routine in order to promote an improvement to infant sleep quality in line with caregiver goals established, for example, as shown at step 204 of FIG. 2. For example, in order to promote an example goal of achieving a more rapid transition by the infant to a sleeping state, the former routine beginning at 7:00 PM of bathing 606, reading 608 and rocking 604 is modified to introduce an action 609 to move the infant from being held to being placed in a crib, with the option of introducing coordinated actions to swaddle the infant 610 and to provide the infant with a pacifier 612 as the infant is being placed in the crib. As a follow-up, a rocking action 604 previously performed by the primary caregiver is replaced with a holding action 616 by the caregiver in combination with action to swaddle the infant 610 and action to provide the pacifier 612. In this manner, actions to alter the behavior of the infant (and, in some cases, to alter the behavior of the caregivers) are introduced in a gradual, non-disruptive manner.

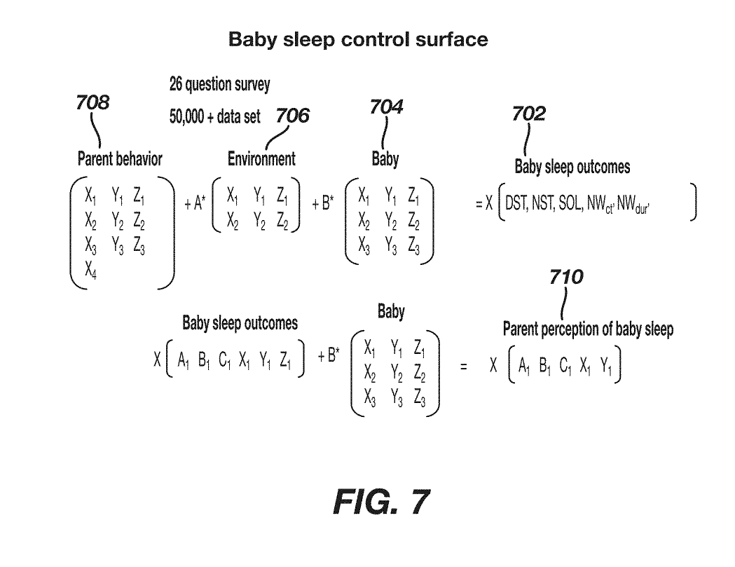

FIG. 7 depicts element of large data set modeling of infant and caregiver behaviors in accordance with aspects of the present disclosure. This large data set may be stored, for example, in the information store 112 of FIG. 1, and interpreted by the remote server 110 in order to select caregiver actions that are correlated with desired infant sleep outcomes. As illustrated in FIG. 7, the large data set may be interrogated by the remote serve 110 of FIG. 1 to determine likely sleep outcomes 702 (for example, including daytime sleep (DST), nighttime sleep (NST), sleep onset latency (SOL), night waking count (NW_{ct}) and night waking duration (NW_{dur})).

Remote server 110 may interrogate the data set to model outcomes 702 as a function of infant biologic conditions 704, infant environmental conditions 706 in proximity to the infant, and caregiver behaviors 708. Caregiver perception of sleep outcomes may also be modeled by the remote server 110 as a function of sleep outcomes 702 and infant biologic conditions 704. As a result of this modeling, the remote server 110 of FIG. 1 can operate caregiver behavior data 708 customization intervention engine 208 of FIG. 2 to select interventions 210 that are tailored to the caregiving tendencies and styles the caregiver and likely to demonstrate the sleep outcomes 702 of FIG. 7 and caregiver perception 710 that are consistent with caregivers goals.

In some aspects, it may be beneficial to provide ongoing and frequent evaluation and feedback to caregiver through Bayesian behavioral methods. The use of these Bayesian (i.e., hypothesis is updated as more information becomes available) methods allows for diagnosis, feedback and intervention in real-time and in non-linear ways. In linear methods, such as a decision tree approach, a series of questions or identifications is navigated one by one, where a first response must be received or acknowledged before a second response can be obtained. Through non-linear methods, interventions and guidance may be provided in a quicker and more robust fashion. Non-linear methods also account for biological changes in the infant as well as the caregiver, such as aging or disease, and also account for cognitive changes whereby the participants learn and modify their own behavior over time.

One method of the present invention uses ongoing and frequent gathering of information, probabilistically determining a most likely diagnosis, and providing feedback. This method includes receiving data, including human behaviors and resultant biological processes. The receipt of this data allows for probabilistic diagnosis and probabilistic determination of high impact questions to be asked or data to be gathered based upon the probability evaluated. This allows for real-time modification of the system, and ongoing reassessment or retargeting of the behavior quantum based upon the frequent tracking. Frequency of tracking or inquiring may be every second, every minute, every hour, every half day, every day, or at other desired intervals.

Care regarding infants sometimes involves rapid change of different mechanisms and therefore it would be helpful to rapidly change and update the problem or goal of a control system. In particular, babies are developing rapidly and tend to change their behaviors on the time scale of days or weeks. Additionally, caregivers are rapidly learning new skills and developing expertise and new perspectives, also often on the time scale of days or weeks. Ideally an effective behavioral control system would update its learning, its data gathering, and/or its interventional recommendations hourly, daily, or weekly.

The action of the control system may be dependent on the process output or result; where feedback from the process variables may be used to alter the control system over time. In this case the action of the control system would be influenced by either ongoing caregiver behavior or the observed baby sleep. A closed loop control system involving a probabilistic determination of problems or goals (e.g., with new set points), which are suggested to and confirmed by the primary user, may be beneficial. For example, in this closed loop system, after a desired goal or problem has been established, the system then initiates a closed loop control process to move toward the desired outcome by establishing and reinforcing behavior change.

The probabilistic determination of the problem or goal of the target individual is then matched to behavioral quantum. The behavioral quantum is understood as involving a discrete and explicit behavioral change packet of action(s) for a particular goal or target outcome that is delivered for implementation at and over a particular time period. This concept is illustrated by FIG. 6B, which depict a series of behavioral quanta (BQ) 621-625 defining actions to be administered by a caregiver over a three-day period. Each of the three days in this period begins with the administration of BQ¹ 621 at approximately 9:30 AM, which may, for example, represent a caregiver intervention to wake an infant. Similarly, BQ² 622 and BQ³ 623 are administered in succession each day, beginning at approximately 7:00 PM. BQ² and BQ³ may, for example, represent caregiver interventions to feed and rock the infant, respectively.

BQ⁴ 624 and BQ⁵ 625 are administered somewhat differently from BQ¹ 621, BQ² 622 and BQ³ 623. On Day 1, BQ⁴ 624 and BQ⁵ 625 are administered in succession starting at about 1:00 AM, On Day 2, the order of administration is reversed (BQ⁵ is administered before BQ⁴), and on Day 3, the start time for the initially-administered BQ⁵ is advanced to 3:00 AM. BQ⁴ and BQ⁵ may, for example, represent caregiver interventions to feed and rock the infant, respectively. With a goal to extend the infant's period of nighttime sleep, BQ⁴ and BQ⁵ may for example represent caregiver interventions to feed and walk the infant, respectively. Beginning on Day 2, the order of administration of BQ⁴ and BQ⁵ is reversed, based on an analysis of data suggesting that initially walking rather than feeding the infant effectively extends the time between successive feedings to promote longer sleep cycles.

Returning to FIG. 7, the aforementioned Bayesian behavioral method may include a closed loop control system, which relies upon continual or repeated monitoring of progress towards a particular goal. As such, the behavioral change toward that goal can be increased or slowed down as needed, or the behavioral quantum can be switched for a new behavioral quantum as necessary. In typical clinical settings, behavioral therapy usually includes open loop control systems, where after a goal is determined a behavioral change is then implemented, but without continual monitoring toward that goal. The probabilistic approach with continual feedback and modification allows the system to be changed and the behavioral quanta to be taken into account to provide for effective intervention. There may be more than one behavioral quanta that may effectively solve an individual problem or aid in the attainment of the individual goal, and also multiple different problems may be solved (or goals reached) by an individual behavior quantum. The behavioral quantum that is recommended to the caregiver can be drawn from an outside goal group without changing the overall goal, and the caregiver need not be made aware of the modification. Such changing may be made during use, at regular and/or irregular intervals, such as daily or biweekly. In this way, novel behavioral therapy techniques can be developed and tailored to a caregiver.

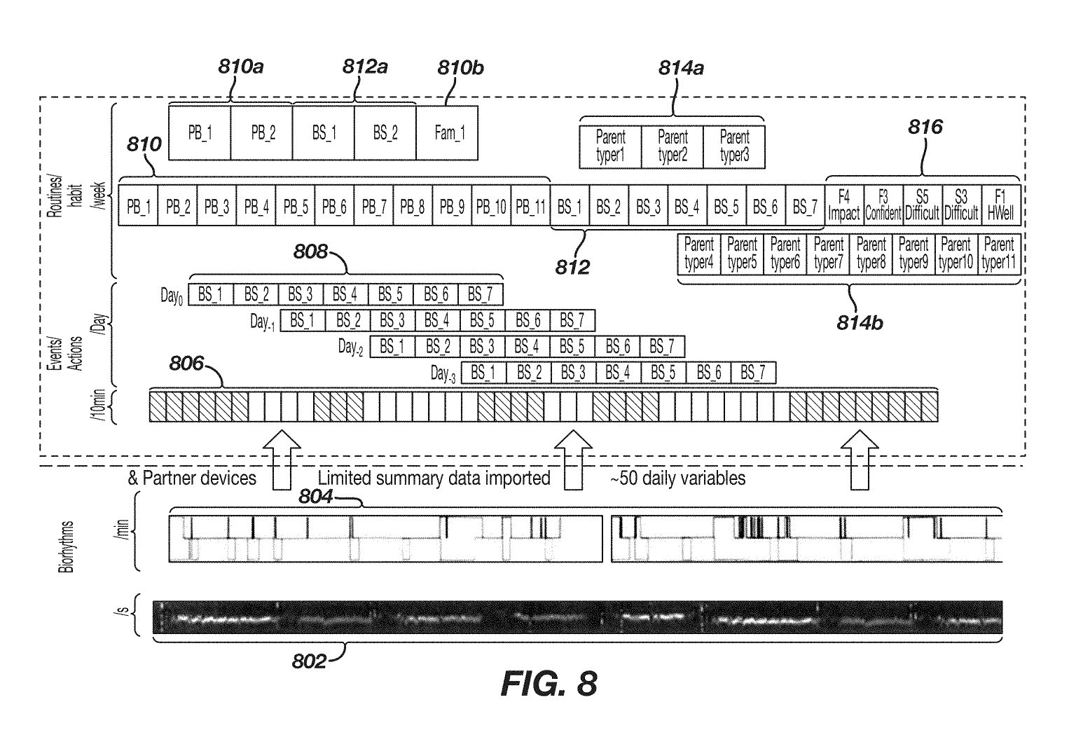

FIG. 8 depicts a data assimilation hierarchy for managing the large data set modeling depicted in FIG. 7. The hierarchy is introduced as a mechanism for quantizing and reducing a large volume of data for a population of infants to a size and form that is suitable for data analysis. With reference for example to FIGS. 4A, 4B and 5, biometric data including biorhythm data 802 is collected for each infant via infant sensors 416a, 518, and indicates a sleep/awake state for an infant. This sleep/awake state information may be sampled and collected by comparators 414, 514 (implemented by server 110 of FIG. 1), for example, at a rate of 12 times per minute. Server 110 is then operative to process this information to produce quantized data 804 that establishes sleep/wake states, for example, at 1 to 5 minute intervals, and then further reduces this data to event data 806 that assigns sleep/wake states in 10 minute intervals.

The event data 806 is analyzed to produce daily summary data 808, which may be characterized for example by seven distinct “baby sleep” variables BS_1 through BS_7, selected for example from among baby sleep parameters 308 as depicted in FIG. 3. Summary data 808 may be accumulated daily for each of a period of days, with the variables BS_1 through BS_7 calculated by the server 110 as a function of one or more of event data 806, quantized data 804, biorhythm data 802 and caregiver data (for example, as provided via sensor(s) 418 and/or survey questions 420 as depicted in FIG. 4A).

Data describing caregiver routines and habits for the infant is also collected and assembled by the server 110. For example, summary data 810 may be accumulated on a weekly basis for parent behavior variables PB_1 through PB_11, selected for example from parent behavior variables 306 as depicted in FIG. 3. Weekly summary data 812 characterizing baby sleep variables BS1_1 through BS_7 may be assembled from the daily summary data 808 characterizing these variables. The most significant data summarizing parent behavior and baby sleep characteristics may be extracted, for example, as parent behavior data 810a and baby sleep data 812a, respectively. From this data, additional data 810b may be prepared for certain “family” variables Fam_1 (for example, including behaviors and trends among multiple infant caregivers and/or multiple infants cared for by a common caregiver).

In addition, parent or caregiver perceptions of infant sleep and caregiving effectiveness may be obtained as summary data 816 (for example, as provided via caregiver surveys 420, 520 as illustrated in FIGS. 4A, 4B and 5). Caregiver surveys may also serve as the source of data for summary data 814a, 814b, for example, characterizing caregiver type traits Parent typer1—Parent typer9. Appendix 1 provides sample survey questions that may be used to assess caregiver type traits Parent typer1—Parent typer9.

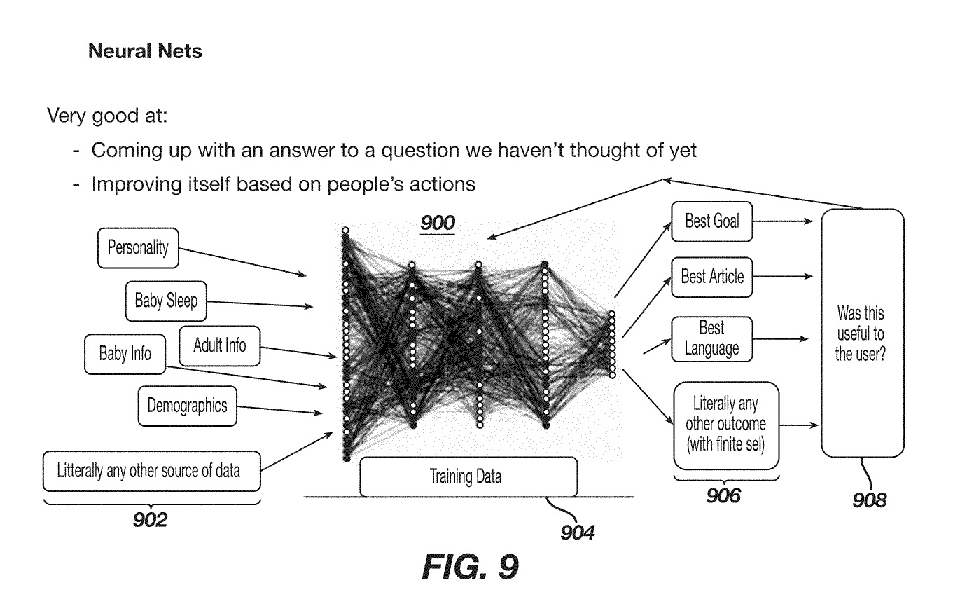

FIG. 9 depicts an analysis engine for analyzing the data described with reference to FIG. 8. The analysis engine is preferably implemented as a neural network 900, which applies at least a portion of the large-scale data set of infant and caregiver information acquired according to the data assimilation hierarchy of FIG. 8. This portion of the data is used as training data 904 for building probabilistic models for determining best infant sleep goals, interventions and outcomes 906 based on baby sleep data 812 and parent behavior data 810 of FIG. 8. Best goals, interventions and outcomes 906 are used, for example, to perform the probabilistic diagnosis 424 and produce the associated intervention plan 426 depicted in FIG. 4B. Feedback 908 based on the effectiveness of intervention plan 426 (for example, as evaluated via survey questionnaires 420 of FIG. 4B) is preferably applied to further train the network 900.

### Introduction: Baby Sleep Score

One primary method to interpret infant sleep is the Brief Infant Sleep Questionnaire (BISQ) for assessment of sleep patterns (0-36 months). Previously BISQ scoring was done via clinical interpretation, but remains difficult given that infant sleep is highly variable and age dependent. We developed and validated a novel scoring method for the infant sleep questionnaire (BISQ-R), and implemented into our digital sleep coaching system to determine clinical efficacy.

### Methods: Baby Sleep Score

To develop the scoring method, we analyzed 33,835 BISQ submissions (1-36 mos; 52.2% boys; 83.0% mothers) from the Johnson's® Bedtime® Sleep App. Three subscales were created for the BISQ-R: Baby Sleep (5 items—night wake duration, sleep onset latency, longest stretch, night wake duration, and total night sleep variables), Parent Perception (3 items), and Parent Behavior (11 items). To normalize each item, continuous data tables (124,500 data points) were generated based on infant age and type of response. Each item was coded to a specific score (0 to 1), and subscales were weight averaged. Implementation and validation of the scoring system was conducted to confirm reliability and convergence.

### Results: Baby Sleep Score

BISQ-R Baby Sleep scores were normalized across all ages, and significantly correlated with Parent Perception scores (R2=0.379, p<0.001). There was a strong logarithmic relationship between Parent Behavior and Baby Sleep, which increased with age (R2=0.91, p<0.001).

### Conclusion: Baby Sleep Score

We developed the BISQ-R, an infant sleep scoring system for clinical, research, and digital use. Construct validity of the BISQ-R resulted in convergent findings between Baby Sleep, Parent Perception, and Parent Behavior scores. The results were published. See R A Gould, J A Mindell, E S Leichman, Russel Michael Walters, “Normalized Scoring System for the Brief Infant Sleep Questionnaire (BISQ)”, April 2018Sleep 41(suppl_1):A285-A285. DOI: 10.1093/sleep/zsy061.765.

### Methods: Digital Sleep System

We next implemented the BISQ-R into our infant digital sleep coaching framework, to clinically validate real-time use of the system. Caregivers of 1200 young children (0-36 months) used the infant digital sleep coaching system (via a free and publicly available smartphone application). We analyzed changes in infant sleep patterns over time, with baseline beginning at day 1 of use, and the final endpoint 30 days later (4-weeks total).

### Results: Digital Sleep System

Overall, Implementation of the BISQ-R within our infant digital sleep coaching system revealed a mean increase of 0.15 (15%) in Baby Sleep score over a 30-day period, compared to baseline (p<0.001). Additionally, Parent Perception of infant sleep outcomes significantly improved, as meaningful results were seen even after only 5 days.

### Conclusion:

Implementation of the BISQ-R within our infant digital sleep coaching system revealed robust clinical efficacy. Both Baby Sleep score and Parental Perception significantly increased over a 30-day period when compared to baseline measurements. The results were published. See R A Gould, J A Mindell, E S Leichman, Russel Michael Walters, “Normalized Scoring System for the Brief Infant Sleep Questionnaire (BISQ)”, April 2018Sleep 41(suppl_1):A285-A285. DOI: 10.1093/sleep/zsy061.765}

### Methods: Digital Sleep System

We next implemented the BISQ-R into our infant digital sleep coaching framework, to clinically validate real-time use of the system. Caregivers of 1200 young children (0-36 months) used the infant digital sleep coaching system (via a free and publicly available smartphone application). We analyzed changes in infant sleep patterns over time, with baseline beginning at day 1 of use, and the final endpoint 30 days later (4-weeks total).

### Results: Digital Sleep System

Overall, Implementation of the BISQ-R within our infant digital sleep coaching system revealed a mean increase of 0.15 (15%) in Baby Sleep score over a 30-day period, compared to baseline (p<0.001). Additionally, Parent Perception of infant sleep outcomes significantly improved, as meaningful results were seen even after only 5 days.

### Conclusion:

Implementation of the BISQ-R within our infant digital sleep coaching system revealed robust clinical efficacy. Both Baby Sleep score and Parental Perception significantly increased over a 30-day period when compared to baseline measurements.

### Results:

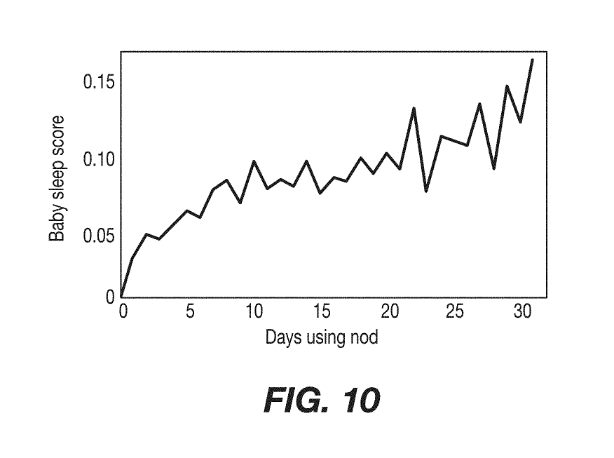

1200 caregivers used our infant digital sleep coaching system for their infant, and progress was tracked over time. Baby Sleep score and Parent Perception was assessed over 30 days, and compared to baseline at day 1. The relative change in baby sleep score is shown in FIG. 10.

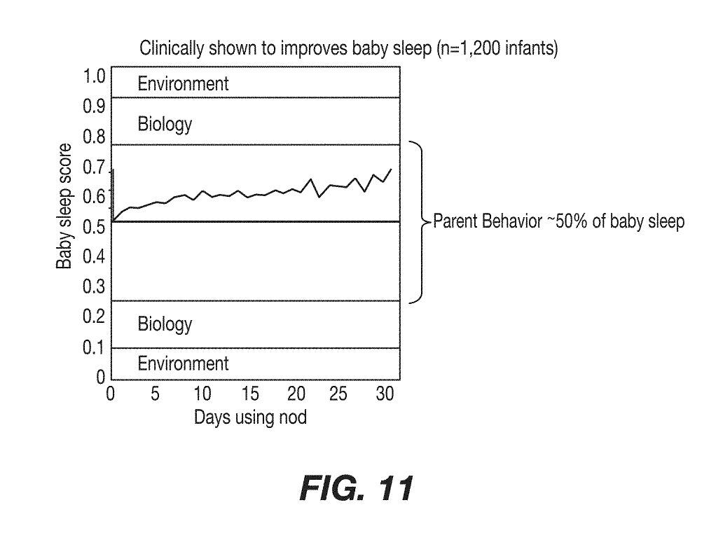

Implementation of the BISQ-R within our infant digital sleep coaching system revealed a mean increase of 0.15 (15%) in Baby Sleep score over a 30-day period, compared to baseline (p<0.001). This is highly significant as parent behavior controls ˜50% of the Baby Sleep score, while other factors such as biology and environment also contribute. The significance of the results is shown in FIG. 11.

### CONCLUSION

It will be understood that, while various aspects of the present disclosure have been illustrated and described by way of example, the invention claimed herein is not limited thereto, but may be otherwise variously embodied according to the scope of the claims presented in this and/or any derivative patent application. It should noted, for example, that although the examples provided in the specification are specificaly directed to caregiver management of infant sleep quality, these same principles may be readily applied to many other caregiver applications. For example, the disclosed invention could additionally be applied managing elder care quality administered in a nursing home or other assisted living facility by a variety of individual caregivers.

We claim:

1. A health care system directed to a caregiver that monitors sleep metrics, patterns and quality for an infant, the system comprising:
a base station in communication with a network;
one or more sensors in communication with the base station, the one or more sensors configured to monitor sleep-relevant characteristics of the infant and environmental conditions in proximity to the infant;
a caregiver communication device in communication with the network; and
a remote server in communication with the network,
wherein the remote server is operative to:
access stored information indicating one or more caregiver typing traits for the caregiver,
receive information from the sensors via the base station indicative of one or more measures of sleep metrics, patterns and quality for the infant,
receive information from the caregiver communication device indicative of a caregiver perception of sleep metrics, patterns and quality for the infant, and
recommend at least one intervention from an array of possible interventions to be acted on for the caregiver, the recommended intervention selected as a function of the one or more caregiver typing traits, the one or more sleep quality measures and the caregiver perception of the sleep metrics, patterns and quality for the infant; and
transmit the recommended intervention to the caregiver communication device.

2. The health care system of claim 1, wherein the one or more sensors comprise biometric sensors for sensing biometric data of the infant.

3. The health care system of claim 1 or claim 2, wherein sensor and question inputs, server and algorithms, communication to the care giver, and care givers interventions constitute a closed loop control system.

4. The health care system of any of the above claims, wherein the one or more biometric sensors are disposed on one or more of a blanket, a mattress or clothing of the infant.

5. The health care system of any of the above claims, wherein the one or more biometric sensors are disposed on one or more of non-contact sensors such as video or radar.

6. The health care system of any of the above claims, wherein at least one of the biometric sensor and the question inputs comprises answers to data entry questions entered through a personal computing device.

7. The health care system of any of the above claims, wherein the one or more sensors comprise environmental sensors.

8. The health care system of claim 7, wherein the one or more environmental sensors monitor one or more of a temperature, light level or sound profile in proximity to the infant.

9. The health care system of any of the above claims, wherein the remote server is further operative to determine the one or more caregiver typing traits as a function of a caregiver survey administered by the remote server.

10. The health care system of any of the above claims, wherein the recommended intervention is further selected as a function of a predetermined sleep quality goal.

11. The health care system of claim 10, wherein the remote server is operative to determine and/or diagnose at least one problem based on the quality goal and the one or more caregiver typing traits, the one or more sleep metric, patterns and quality measures and the caregiver perception of the sleep quality for the infant, and the recommended intervention is identified as impacting the at least one problem.

12. The health care system of any of the above claims, wherein the system is directed to a plurality of caregivers that monitor sleep quality for the infant, and the remote server is operative to recommend at least one action to each of the plurality of caregivers as a function of the caregiver typing traits of the respective caregiver.

13. The health care system of any of the above claims, wherein the remote server is further configured to select one or more coaching suggestions to be provided to the caregiver in association with the recommended action, the one or more coaching suggestions being selected as a function of the caregiver typing traits.

14. The health care system of claim 13, wherein the one or more coaching suggestions are selected from the group consisting of reminder messages, encouragement messages, alarms, and environmental changes in proximity to the caregiver.

15. The health care system of any of the above claims, further comprising:
an environmental control device for controlling one or more of the temperature, light level or sound profile in proximity to the infant,
wherein the remote server is further configured to:
recommend at least on environmental change based on one or more of the one or more caregiver typing traits, the one or more sleep quality measures and the caregiver perception of the sleep quality for the infant, and
transmit the recommended environmental change to the environmental control device.

16. The health care system of claim 15, wherein the remote server is further operative to recommend a sequence of caregiver actions and environmental changes over the course of a day, the sequence defining a daily routine for the infant.

17. The health care system of claim 15 or claim 16, wherein the remote server is operative to alter at least one of the sequence of caregiver actions and environmental changes, and recommend the altered sequence over the course of a subsequent day.

18. The healthcare system of any of the above claims, wherein the remote server is operative to:
confirm that the recommended caregiver action was applied;
receive updated information from the sensors indicative of one or more measures of a current sleep quality for the infant,
receive updated information from the caregiver communication device indicative of a current caregiver perception of sleep quality for the infant,
receive an updated caregiver perception of the sleep quality for the infant;
evaluate the effectiveness of the recommended action in improving to caregiver perception of sleep quality.

19. The healthcare system of any of claims 15-17, wherein the remote server is operative to:
confirm that the environmental change was applied;
receive updated information from the sensors indicative of one or more measures of a current sleep quality for the infant,
receive updated information from the caregiver communication device indicative of a current caregiver perception of sleep quality for the infant,
receive an updated caregiver perception of the sleep quality for the infant;
evaluate the effectiveness of the environmental change in improving to caregiver perception of sleep quality.

20. A method for directing a caregiver that monitors sleep quality for an infant, the system comprising the steps of:
monitoring one or more sensors for sleep-relevant characteristics of the infant and environmental conditions in proximity to the infant;
accessing stored information indicating one or more caregiver typing traits for the caregiver;
accessing information from a caregiver communication device indicative of a caregiver perception of sleep quality for the infant;
recommending at least one action for the caregiver, the recommended action selected as a function of the one or more caregiver typing traits, the one or more sleep quality measures and the caregiver perception of the sleep quality for the infant; and
transmitting the recommended action to the caregiver communication device

21. The health care system of claim 11, wherein the determination and/or diagnosis produces a probabilistic analysis of a plurality of potential problems based on the quality goal and the one or more caregiver typing traits.

22. The health care system of claim 21, wherein the probabilistic analysis is based on quality goal, caregiver typing trait, sleep patterns, sleep metrics, quality measure, caregiver perception and caregiver intervention information for a population of caregivers and infants.

23. The health care system of claim 21, wherein the probabilistic analysis provides recommended caregiver goals based on the potential problems.

24. The health care system of claim 11 or claim 21, wherein the determination and/or diagnosis is conducted approximately daily.

25. The health care system of any of the above claims, wherein the recommended intervention is associate with a set of behavioral quanta.

26. The health care system of any of claim 11, 21 or 24, wherein the probabilistic analysis is performed by a trained neural network,
